(19) United States
(12) Patent Application Publication
(10) Pub. No.: US 2022/0306704 A1
(43) Pub. Date: Sep. 29, 2022

(54) NEUROTOXINS FOR USE IN INHIBITING CGRP

(71) Applicant: Bonti, Inc., North Chicago, IL (US)

(72) Inventors: Michael Jarpe, North Chicago, IL (US); Fauad Hasan, North Chicago, IL (US)

(21) Appl. No.: 17/654,226

(22) Filed: Mar. 9, 2022

Related U.S. Application Data
(63) Continuation of application No. 16/374,378, filed on Apr. 3, 2019, now abandoned.
(60) Provisional application No. 62/651,839, filed on Apr. 3, 2018.

(51) Int. Cl. C07K 14/33; A61K 38/16; A61P 31/04; A61P 17/02; A61K 9/00; A61P 41/00; A61K 38/48; A61P 31/00

(52) U.S. Cl. CPC C07K 14/33; A61K 38/164; A61P 31/04; A61P 17/02; A61K 9/0019; A61K 9/0014; A61P 41/00; A61K 38/4893; A61K 9/0024; A61P 31/00

(57) ABSTRACT

Disclosed herein are compositions and methods for use in inhibiting CGRP production and release.

### CROSS REFERENCE TO RELATED APPLICATIONS

This application is a continuation of U.S. patent application Ser. No. 16/374,378, filed on Apr. 3, 2019, which claims the benefit of priority to U.S. Provisional Patent Application No. 62/651,839, filed on Apr. 3, 2018, which is incorporated herein by reference in its entirety.

### TECHNICAL FIELD

The present disclosure relates to the use of neurotoxins to treat disorders.

### BACKGROUND

Calcitonin gene-related peptide (CGRP) is a member of the calcitonin family of peptides, which in humans exists in two forms, α-CGRP and β-CGRP. α-CGRP is a 37-amino acid peptide and is formed from the alternative splicing of the calcitonin/CGRP gene located on chromosome 11. The less-studied β-CGRP differs in three amino acids and is encoded in a separate gene in the same vicinity. CGRP is produced in both peripheral and central neurons. It is a potent peptide vasodilator. In the spinal cord, the function and expression of CGRP may differ depending on the location of synthesis. CGRP is derived mainly from the cell bodies of motor neurons when synthesized in the ventral horn of the spinal cord and may contribute to the regeneration of nervous tissue after injury. CGRP is also derived from dorsal root ganglion when synthesized in the dorsal horn of the spinal cord. In the trigeminal vascular system, the cell bodies on the trigeminal ganglion are the main source of CGRP. CGRP is thought to play a role in cardiovascular homeostasis and nociception.

CGRP mediates its effects through a heteromeric receptor composed of a G protein-coupled receptor called calcitonin receptor-like receptor (CALCRL) and a receptor activity-modifying protein (RAMP1). CGRP receptors are found throughout the body, suggesting that the protein may modulate a variety of physiological functions in all major systems (e.g., respiratory, endocrine, gastrointestinal, immune, and cardiovascular). The extracellular loop number 2 is fundamental for ligand induced activation, with key interactions of R274/Y278/D280/W283. An aspect of the interaction between the sensory and immune systems is the release of CGRP.

Preclinical evidence suggests that, during a migraine, activated primary sensory neurons (meningeal nociceptors) in the trigeminal ganglion release CGRP from their peripherally projecting nerve endings located within the meninges. This CGRP then binds to and activates CGRP receptors located around meningeal vessels, causing vasodilation, mast cell degranulation, and plasma extravasation. Human observations have further implicated the role of CGRP in the pathophysiology of migraine. Activation of primary sensory neurons in the trigeminal vascular system in humans can cause the release of CGRP. During some migraine attacks, increased concentrations of CGRP can be found in both saliva and plasma drawn from the external jugular vein. Furthermore, intravenous administration of alpha-CGRP can induce headache in individuals susceptible to migraine.

Infection is the invasion of an organism's body tissues by disease-causing agents, their multiplication, and the reaction of host tissues to the agents and the toxins they produce. Infections can be caused by viruses, viroids, prions, bacteria, nematodes such as parasitic roundworms and pinworms, arthropods such as ticks, mites, fleas, and lice, fungi such as ringworm, and other macro-parasites such as tapeworms and other helminths. Symptomatic infections are apparent and clinical, whereas an infection that is active but does not produce noticeable symptoms may be called in-apparent, silent, subclinical, or occult. An infection that is inactive or dormant is called a latent infection; an example of a latent bacterial infection is latent tuberculosis. Some viral infections can also be latent; examples of latent viral infections are any of those from the Herpesviridae family. A short-term infection is an acute infection. A long-term infection is a chronic infection. Infections can be further classified by causative agent (bacterial, viral, fungal, parasitic), and by the presence or absence of systemic symptoms (sepsis).

Mammalian hosts react to infections with an innate response, often involving inflammation, followed by an adaptive response. Specific medications used to treat infections include antibiotics, antivirals, antifungals, anti-protozoals, and anti-helminthics. Infectious diseases resulted in 9.2 million deaths in 2013 (about 17% of all deaths).

Pain is a distressing feeling often caused by intense or damaging stimuli. The International Association for the Study of Pain's widely-used definition defines pain as “an unpleasant sensory and emotional experience associated with actual or potential tissue damage, or described in terms of such damage,” however, due to it being a complex, subjective phenomenon, defining pain has been a challenge. In medical diagnosis, pain is regarded as a symptom of an underlying condition. Pain motivates the individual to withdraw from damaging situations, to protect a damaged body part while it heals, and to avoid similar experiences in the future. Most pain resolves once the noxious stimulus is removed and the body has healed, but it may persist despite removal of the stimulus and apparent healing of the body. Sometimes pain arises in the absence of any detectable stimulus, damage or disease.

Both pain and inflammation are protective responses. However, these self-limiting conditions (with well-established negative feedback loops) become pathological if left uncontrolled. Both pain and inflammation can interact with each other in a multi-dimensional manner, for example via peripheral, sensory and central nervous system levels. Innate immunity plays a critical role in central sensitization and in establishing acute pain as chronic condition. Moreover, inflammatory mediators also exhibit psychological effects, thus contributing towards the emotional elements associated with pain. However, there is also a considerable anti-inflammatory and analgesic role of immune system.

A nociceptor is a type of receptor at the end of a sensory neuron's axon that responds to damaging or potentially damaging stimuli by sending “possible threat” signals to the spinal cord and the brain. Nociceptor neurons densely innervate peripheral barrier tissues that are exposed to pathogens: If the brain thinks the threat is credible, it creates the sensation of pain to direct attention to the body part, so the threat can hopefully be mediated. This process is called nociception. In mammals, nociceptors are found in any area of the body that can sense noxious stimuli. External nociceptors are found in tissue such as the skin (cutaneous nociceptors), the corneas, and the mucosa. Internal nociceptors are found in a variety of organs, such as the muscles, the joints, the bladder, the gut, and the digestive tract. The cell bodies of these neurons are located in either the dorsal root ganglia or the trigeminal ganglia. The trigeminal ganglia are specialized nerves for the face, whereas the dorsal root ganglia are associated with the rest of the body. The axons extend into the peripheral nervous system and terminate in branches to form receptive fields.

### SUMMARY

Disclosed herein are compositions and methods for use in minimizing scarring. For example, disclosed embodiments comprise use of a “fast-acting” botulinum toxin to reduce muscle tension in the proximity of a wound, thus preventing or reducing scarring.

Botulinum neurotoxins can effectively reduce or block neuronal release of CGRP, for example during infection, and can therefore be useful in the treatment of a number of conditions. For example, disclosed embodiments can limit or prevent bacterial infections, for example necrotizing lesions, for example those caused by S. Pyogenes. Disclosed embodiments can reduce or prevent pain.

In a first aspect, a method for treating infection is provided. In one embodiment, the method comprises treating necrotizing lesions caused by bacterial infection. In one embodiment, the method comprises administering a therapeutically effective amount of a fast-acting neurotoxin to a patient in the proximity of a lesion caused by the infection.

In another aspect, a method for treating pain in a patient in need thereof by inhibiting cGRP production or release. The method comprises administering a therapeutically effective amount of a fast-acting neurotoxin to an area where the patient is experiencing pain.

In another aspect, a method for reducing the occurrence of pain in a patient in need thereof by inhibiting cGRP production or release. The method comprises administering a therapeutically effective amount of a fast-acting and/or short-duration neurotoxin to an area where the patient has experienced pain and/or is likely to experience pain.

In one embodiment, a method for reducing post-operative pain in a patient in need thereof is provided, the method comprises locally administering a therapeutically effective amount of a fast-acting and/or short-duration neurotoxin in the proximity of the area of a surgical incision.

In another embodiment, a method for reducing the occurrence of post-operative pain is provided, the method comprises administering a therapeutically effective amount of a fast-acting and/or short-duration neurotoxin in the proximity of the area of a surgical incision.

In one embodiment, the fast-acting neurotoxin comprises botulinum neurotoxin serotype E.

In some embodiments, the administering is performed before a surgical procedure. In other embodiments, the administering is performed during a surgical procedure. In yet other embodiments, the administering is performed after a surgical procedure.

In one embodiment, the therapeutically effective amount comprises an amount of between about 10⁻³ U/kg and about 35 U/kg. In another embodiment, the therapeutically effective amount comprises an amount of between about 1 U/kg and about 25 U/kg. In still another embodiment, the therapeutically effective amount comprises an amount of between about 5 U/kg and about 15 U/kg.

In yet another embodiment, the therapeutically effective amount comprises an amount of between about 0.2 nanograms and about 2 nanograms.

In another embodiment, muscle activity in the proximity of a skin incision or laceration is reduced, thus reducing or preventing scar formation.

In another embodiment, the botulinum toxin is a fast-recovery toxin.

In yet another embodiment, the “fast-acting” botulinum toxin is also a fast-recovery toxin.

In still another embodiment, the method further comprises administration of a fast-acting botulinum neurotoxin in combination with, for example, a slower-acting neurotoxin. In one embodiment, the slower-acting neurotoxin is botulinum toxin subtype A (BoNT/A).

In other embodiments, the method further comprises administration of a fast-recovery botulinum neurotoxin in combination with, for example, a slower-recovery neurotoxin.

In another embodiment, the neurotoxin is administered at a dose below that which would cause muscle paralysis.

The fast onset of muscle relaxing effect with BoNT/E offers improved healing, reduced post-operative pain and reduced side effects in subjects, such as muscle stiffness, eye ptosis or neck weakness. The shorter duration of muscle relaxation (2-4 weeks) offered by BoNT/E is also desirable compared to 3-4 months with BoNT/A products, as BoNT/E may allow faster recovery and rehabilitation post-operatively.

In other embodiments, the formulation or composition comprising BoNT/E is administered intramuscularly.

### BRIEF DESCRIPTION OF THE DRAWINGS

FIG. 1 shows a depiction of the primary structure of a botulinum neurotoxin (BoNT).

FIGS. 2A-2D are schematic and ribbon representations of BoNT/E (FIGS. 2A, 2C) and BoNT/B (FIGS. 2B, 2D). The catalytic, translocation, and binding domains are labeled CD, TD, and BD, respectively.

FIGS. 3A-3B are illustrations of the human body, anterior view (FIG. 3A) and posterior view (FIG. 3B), identifying the muscles.

FIG. 4 is an illustration of the human body with the nerves identified.

FIG. 5 shows forehead injection sites for a study described in Example 1.

FIG. 6 is a bar graph showing the % IR-2 responders (primary efficacy outcome) of the subjects in the study described in Example 1.

FIG. 7 is a bar graph showing the proportions of subjects in Example 1 with investigator-assessed FWS grades of none or mild GL at maximum frown.

FIG. 8 is a bar graph showing the effect of a single local administration of a representative fast-acting toxin, BoNT/E, in a rat model of Post-operative pain.

### DETAILED DESCRIPTION

Embodiments disclosed herein can effectively block neuronal release of CGRP, for example during infection, pain, inflammation, and can therefore be useful in the treatment of a number of conditions. For example, disclosed embodiments can reduce or prevent pain. Disclosed embodiments can reduce inflammation. Disclosed embodiments can limit or prevent bacterial necrotizing lesions, for example those caused by S. pyogenes.

There are several ways to categorize pain. One is to separate it into acute pain and chronic pain. Acute pain typically comes on suddenly and has a limited duration. It's frequently caused by damage to tissue such as bone, muscle, or organs, and the onset is often accompanied by anxiety or emotional distress. Chronic pain lasts longer than acute pain and is generally somewhat resistant to medical treatment. It's usually associated with a long-term illness, such as osteoarthritis. In some cases, such as with fibromyalgia, it's one of the defining characteristic of the disease. Chronic pain can be the result of damaged tissue, but very often is attributable to nerve damage.

Exemplary types of pain suitable for treatment using disclosed compositions and methods include nociceptive, neuropathic, and inflammatory pain.

Nociceptive represents the normal response to noxious insult or injury of tissues such as skin, muscles, visceral organs, joints, tendons, or bones. Examples include: (a) Somatic-musculoskeletal (joint pain, myofascial pain), cutaneous; often well localized; and (b) Visceral-hollow organs and smooth muscle.

Embodiments disclosed herein comprise compositions and methods for treating nociceptive pain.

Neuropathic pain is initiated or caused by a primary lesion or disease in the somatosensory nervous system. Sensory abnormalities range from deficits perceived as numbness to hypersensitivity (hyperalgesia or allodynia), and to paresthesias such as tingling. Examples include, but are not limited to, diabetic neuropathy, postherpetic neuralgia, spinal cord injury pain, phantom limb (post-amputation) pain, and post-stroke central pain.

Embodiments disclosed herein comprise compositions and methods for treating neuropathic pain.

Inflammatory pain is a result of activation and sensitization of the nociceptive pain pathway by a variety of mediators released at a site of tissue inflammation. The mediators that have been implicated as key players are proinflammatory cytokines such IL-1-alpha, IL-1-beta, IL-6 and TNF-alpha, chemokines, reactive oxygen species, vasoactive amines, lipids, ATP, acid, and other factors released by infiltrating leukocytes, vascular endothelial cells, or tissue resident mast cells. Examples include appendicitis, rheumatoid arthritis, inflammatory bowel disease, and herpes zoster.

Embodiments disclosed herein comprise compositions and methods for treating inflammatory pain.

Clinical implications of classification: Pathological processes never occur in isolation and consequently more than one mechanism may be present, and more than one type of pain may be detected in a single patient; for example, it is known that inflammatory mechanisms are involved in neuropathic pain. There are well-recognized pain disorders that are not easily classifiable. Our understanding of their underlying mechanisms is still rudimentary though specific therapies for those disorders are well known; they include cancer pain, migraine and other primary headaches and wide-spread pain of the fibromyalgia type.

Pain Intensity: Can be broadly categorized as: mild, moderate and severe. It is common to use a numeric scale to rate pain intensity where 0=no pain and 10 is the worst pain imaginable:
a. Moderate: 5/10 to 6/10
b. Moderate: 5/10 to 6/10
c. Severe: ≥7/10

Embodiments disclosed herein comprise compositions and methods for treating mild, moderate or severe pain.

Time course: Pain duration
a. Acute pain: pain of less than 3 to 6 months duration
b. Chronic pain: pain lasting for more than 3-6 months, or persisting beyond the course of an acute disease, or after tissue healing is complete.
c. Acute-on-chronic pain: acute pain flare superimposed on underlying chronic pain.

Embodiments disclosed herein comprise compositions and methods for treating mild, moderate or severe acute, chronic, or acute-on-chronic pain.

Disclosed embodiments can comprise treatment of somatic pain, which is typically pain caused by the activation of pain receptors in either the body surface or musculoskeletal tissues.

Disclosed embodiments can comprise treatment of visceral pain, resulting when internal organs are damaged or injured. Visceral pain is caused by the activation of pain receptors in the chest, abdomen or pelvic areas. Visceral pain is often vague and not well localized and is usually described as pressure-like, deep squeezing, dull or diffuse. Visceral pain can be caused by problems with internal organs, such as the stomach, kidney, gallbladder, urinary bladder, and intestines. Visceral pain can also be caused by problems with abdominal muscles and the abdominal wall, such as spasm.

Disclosed embodiments can comprise treatment of neuropathic pain caused by injury or malfunction to the spinal cord and/or peripheral nerves. Neuropathic pain is typically a burning, tingling, shooting, stinging, or “pins and needles” sensation. This type of pain usually occurs within days, weeks, or months of the injury and tends to occur in waves of frequency and intensity. Neuropathic pain is diffuse and occurs at the level or below the level of injury, most often in the legs, back, feet, thighs, and toes, although it can also occur in the buttocks, hips, upper back, arms, fingers, abdomen, and neck.

Embodiments can be used to treat, for example, headache pain, toothache pain, and the like.

Disclosed embodiments can interrupt immune system pathways, for example the inflammation pathway.

Disclosed embodiments can comprise treatment of infections, for example bacterial or fungal infections.

Necrotizing soft tissue infections are a broad category of bacterial and fungal skin infections. Descriptive terms vary based on the location, depth, and extent of infection (e.g., Fournier's gangrene [necrotizing perineal infection], necrotizing fasciitis [deep subcutaneous infection]). Depending on the depth of invasion, necrotizing soft tissue infections can cause extensive local tissue destruction, tissue necrosis, systemic toxicity, and even death. Despite surgical advances and the introduction of antibiotics, reported mortality rates for necrotizing soft tissue infections range from 6 percent to as high as 76 percent.

Disclosed herein are methods and compositions for treating necrotizing soft tissue infections. Embodiments disclosed herein can effectively limit or block neuronal release of CGRP during infection and limit or prevent bacterial necrotizing lesions. For example, disclosed embodiments can comprise administering disclosed compositions in proximity to bacterial necrotizing lesions, for example those caused by S. pyogenes.

Disclosed embodiments can comprise administration of doses lower than the dose required to inhibit or prevent muscle contraction.

In some embodiments, compositions disclosed herein can comprise fast-acting botulinum toxins, for example, botulinum type E.

In some embodiments, compositions disclosed herein can comprise fast-recovery botulinum toxins, for example, botulinum type E.

In some embodiments, compositions disclosed herein can comprise fast acting, fast-recovery botulinum toxins, for example, botulinum type E.

### Definitions

“Administration,” or “to administer” means the step of giving (i.e. administering) a pharmaceutical composition or active ingredient to a subject. The pharmaceutical compositions disclosed herein can be administered via a number of appropriate routes. For example, intramuscular, intradermal, subcutaneous administration, intrathecal administration, intraperitoneal administration, topical (transdermal), instillation, and implantation (for example, of a slow-release device such as polymeric implant or miniosmotic pump) can all be appropriate routes of administration.

“Alleviating” means a reduction in the occurrence of a pain, of a headache, or of any symptom or cause of a condition or disorder. Thus, alleviating includes some reduction, significant reduction, near total reduction, and total reduction.

The term “amino acid” means a naturally occurring or synthetic amino acid, as well as amino acid analogs, stereoisomers, and amino acid mimetics that function similarly to the naturally occurring amino acids. Included by this definition are natural amino acids such as: (1) histidine (His; H) (2) isoleucine (Ile; I) (3) leucine (Leu; L) (4) Lysine (Lys; K) (5) methionine (Met; M) (6) phenylalanine (Phe; F) (7) threonine (Thr; T) (8) tryptophan (Trp; W) (9) valine (Val; V) (10) arginine (Arg; R) (11) cysteine (Cys; C) (12) glutamine (Gln; Q) (13) glycine (Gly; G) (14) proline (Pro; P) (15) serine (Ser; S) (16) tyrosine (Tyr; Y) (17) alanine (Ala; A) (18) asparagine (Asn; N) (19) aspartic acid (Asp; D) (20) glutamic acid (Glu; E) (21) selenocysteine (Sec; U); including unnatural amino acids: (a) citrulline (Cit); (b) cystine; (c) gama-amino butyric acid (GABA); (d) ornithine (Orn); (f) theanine; (g) homocysteine (Hey); (h) thyroxine (Thx); and amino acid derivatives such as betaine; carnitine; carnosine creatine; hydroxytryptophan; hydroxyproline (Hyp); N-acetyl cysteine; S-Adenosyl methionine (SAM-e); taurine; tyramine.

“Animal protein free” means the absence of blood derived, blood pooled and other animal derived products or compounds. “Animal” means a mammal (such as a human), bird, reptile, fish, insect, spider or other animal species. “Animal” excludes microorganisms, such as bacteria. Thus, an animal protein free pharmaceutical composition can include a botulinum neurotoxin. For example, an “animal protein free” pharmaceutical composition means a pharmaceutical composition which is either substantially free or essentially free or entirely free of a serum derived albumin, gelatin and other animal derived proteins, such as immunoglobulins. An example of an animal protein free pharmaceutical composition is a pharmaceutical composition which comprises or which consists of a botulinum toxin (as the active ingredient) and a suitable polysaccharide as a stabilizer or excipient.

“Botulinum toxin” or “botulinum neurotoxin” means a neurotoxin produced by Clostridium botulinum, as well as a botulinum toxin (or the light chain or the heavy chain thereof) made recombinantly by a non-Clostridial species. The phrase “botulinum toxin”, as used herein, encompasses the botulinum toxin serotypes A, B, C, D, E, F, G, H and X, and their subtypes, mosaic toxins, such as BoNT/DC and BoNT/CD, and any other types of subtypes thereof, or any re-engineered proteins, analogs, derivatives, homologs, parts, sub-parts, variants or versions, in each case, of any of the foregoing. “Botulinum toxin”, as used herein, also encompasses a “modified botulinum toxin”. Further “botulinum toxin” as used herein also encompasses a botulinum toxin complex, (for example, the 300, 600 and 900 kDa complexes), as well as the neurotoxic component of the botulinum toxin (150 kDa) that is unassociated with the complex proteins. “Purified botulinum toxin” means a pure botulinum toxin or a botulinum toxin complex that is isolated, or substantially isolated, from other proteins and impurities which can accompany the botulinum toxin as it is obtained from a culture or fermentation process. Thus, a purified botulinum toxin can have at least 95%, and more preferably at least 99% of the non-botulinum toxin proteins and impurities removed.

“Clostridial neurotoxin” refers to any toxin produced by a Clostridial toxin strain that can execute the overall cellular mechanism whereby a Clostridial toxin intoxicates a cell and encompasses the binding of a Clostridial toxin to a low or high affinity Clostridial toxin receptor, the internalization of the toxin/receptor complex, the translocation of the Clostridial toxin light chain into the cytoplasm and the enzymatic modification of a Clostridial toxin substrate. Non-limiting examples of Clostridial toxins include Botulinum toxins, such as a BoNT/A, a BoNT/B, a BoNT/C₁, a BoNT/D, a BoNT/CD, a BoNT/DC, a BoNT/E, a BoNT/F, a BoNT/G, a BoNT/H (also known as type FA or HA), a BoNT/X, a Tetanus toxin (TeNT), a Baratii toxin (BaNT), and a Butyricum toxin (BuNT). The BoNT/C₂ cytotoxin and BoNT/C₃ cytotoxin, not being neurotoxins, are excluded from the term “Clostridial toxin.” The term Clostridial toxin also includes the approximately 150-kDa Clostridial toxin alone (i.e. without the NAPs). A Clostridial toxin includes naturally occurring Clostridial toxin variants, such as, e.g., Clostridial toxin isoforms and Clostridial toxin subtypes; non-naturally occurring Clostridial toxin variants, such as, e.g., conservative Clostridial toxin variants, non-conservative Clostridial toxin variants, Clostridial toxin chimeric variants and active Clostridial toxin fragments thereof, or any combination thereof. A Clostridial toxin also includes Clostridial toxin complexes, which refers to a complex comprising a Clostridial toxin and non-toxin associated proteins (NAPs), such as, e.g., a Botulinum toxin complex, a Tetanus toxin complex, a Baratii toxin complex, and a Butyricum toxin complex. Non-limiting examples of Clostridial toxin complexes include those produced by a Clostridium botulinum, such as, e.g., a 900-kDa BoNT/A complex, a 500-kDa BoNT/A complex, a 300-kDa BoNT/A complex, a 500-kDa BoNT/B complex, a 500-kDa BoNT/C₁ complex, a 500-kDa BoNT/D complex, a 300-kDa BoNT/D complex, a 300-kDa BoNT/E complex, and a 300-kDa BoNT/F complex.

“Effective amount” as applied to the biologically active ingredient means that amount of the ingredient which is generally sufficient to affect a desired change in the subject. For example, where the desired effect is a reduction of scar formation, an effective amount of the ingredient is that amount which causes at least a substantial reduction of scar formation, and without resulting in significant toxicity. In other aspects of this embodiment, a therapeutically effective concentration of a Clostridial toxin active ingredient reduces a symptom associated with the aliment being treated by, e.g., at most 10%, at most 20%, at most 30%, at most 40%, at most 50%, at most 60%, at most 70%, at most 80%, at most 90% or at most 100%.

“Fast-acting” as used herein refers to a botulinum toxin that produces effects in the patient more rapidly than those produced by, for example, a botulinum neurotoxin type A, such as onabotulinumtoxinA. For example, the effects of a fast-acting botulinum toxin can be produced within 12 hours, 24 hours or 36 hours. Thus, relative to a fast-acting toxin such as type E, botulinum toxin type A can be classified as a “slower-acting” botulinum toxin. In some cases, a slower-acting botulinum toxin is referred to as an “intermediate-acting” toxin. In some embodiments, fast-acting botulinum toxin produces a measurable therapeutic effect within 6 hours, 12 hours, 24 hours or 36 hours after its administration, and/or its effect is observed at least about 50% sooner than the therapeutic effect produced by onabotulinumtoxinA.

“Fast-recovery” as used herein refers to a botulinum toxin whose effects diminish in a patient more rapidly than those produced by, for example, a botulinum neurotoxin type A, such as onabotulinumtoxinA. In some embodiments, the therapeutic effect of the fast-recovery botulinum toxin diminishes within about 3 months, 2 months or 6 weeks after its administration, and its effects diminish about 50% sooner than the effects produced by onabotulinumtoxinA. For example, the effects of a fast-recovery botulinum toxin can diminish within, for example, 120 hours, 150 hours, 300 hours, 350 hours, 400 hours, 500 hours, 600 hours, 700 hours, 800 hours, or the like. It is known that botulinum toxin type A can have an efficacy for up to 12 months (European J. Neurology 6 (Supp 4): S111-S1150:1999), and in some circumstances for as long as 27 months, when used to treat glands, such as in the treatment of hyperhydrosis. See e.g. Bushara K., Botulinum toxin and rhinorrhea, Otolaryngol Head Neck Surg 1996; 114(3):507, and The Laryngoscope 109:1344-1346:1999. However, the usual duration of an intramuscular injection of a botulinum neurotoxin type A is typically about 3 to 4 months. Thus, relative to a fast-recovery toxin such as type E, botulinum toxin type A, such as onabotulinumtoxinA, can be classified as a “slower-recovery” or “longer acting” botulinum toxin.

“Intermediate-acting” as used herein refers to a botulinum toxin that produces effects more slowly than would a fast-acting toxin.

“Local administration” means direct administration of a pharmaceutical at or to the vicinity of a site on or within an animal body, at which site a biological effect of the pharmaceutical is desired, such as via, for example, intramuscular or intra- or subdermal injection or topical administration. Local administration excludes systemic routes of administration, such as intravenous or oral administration. Topical administration is a type of local administration in which a pharmaceutical agent is applied to a patient's.

“Neurotoxin” means a biologically active molecule with a specific affinity for a neuronal cell surface receptor. Neurotoxin includes Clostridial toxins both as pure toxin and as complexed with one to more non-toxin, toxin associated proteins.

“Patient” means a human or non-human subject receiving medical or veterinary care.

“Pharmaceutical composition” means a composition comprising an active pharmaceutical ingredient, such as, for example, a Clostridial toxin active ingredient such as a botulinum toxin, and at least one additional ingredient, such as, for example, a stabilizer or excipient or the like. A pharmaceutical composition is therefore a formulation which is suitable for diagnostic or therapeutic administration to a subject, such as a human patient. The pharmaceutical composition can be, for example, in a lyophilized or vacuum dried condition, a solution formed after reconstitution of the lyophilized or vacuum dried pharmaceutical composition, or as a solution or solid which does not require reconstitution. As stated, a pharmaceutical composition can be liquid or solid. A pharmaceutical composition can be animal-protein free.

“Preparing” a surgical site refers to administering a composition disclosed herein to reduce muscle tension in the incision area.

“Supplemental administration” as used herein refers to a botulinum administration that follows an initial neurotoxin administration.

“Therapeutic formulation” means a formulation that can be used to treat and thereby alleviate a disorder, or a disease and/or symptom associated thereof, such as a disorder or a disease characterized by an activity of a peripheral muscle.

“Therapeutically effective concentration”, “therapeutically effective amount,” “effective amount,” “effective dose,” and “therapeutically effective dose” refer to the minimum dose of an agent (e.g. such as a botulinum toxin or pharmaceutical composition comprising botulinum toxin) needed to achieve the desired therapeutic effect and includes a dose sufficient to reduce a symptom associated with a disease, disorder or condition being treated without causing significant negative or adverse side effects.

“Treat,” “treating,” or “treatment” means an alleviation or a reduction (which includes some reduction, a significant reduction a near total reduction, and a total reduction), resolution or prevention (temporarily or permanently) of an disease, disorder or condition, so as to achieve a desired therapeutic or cosmetic result, such as by healing of injured or damaged tissue, or by altering, changing, enhancing, improving, ameliorating and/or beautifying an existing or perceived disease, disorder or condition.

“Unit” or “U” means an amount of active botulinum neurotoxin standardized to have equivalent neuromuscular blocking effect as a Unit of commercially available botulinum neurotoxin type A.

“Wound” as used herein refers to a disruption to the skin, for example caused by injury or intentionally.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present specification should be construed as indicating any non-claimed element essential to the practice of embodiments disclosed herein.

### Neurotoxin Compositions

Embodiments disclosed herein comprise neurotoxin compositions, for example fast-recovery neurotoxins. Such neurotoxins can be formulated in any pharmaceutically acceptable formulation in any pharmaceutically acceptable form. The neurotoxin can also be used in any pharmaceutically acceptable form supplied by any manufacturer.

The neurotoxin can be made by a Clostridial bacterium, such as by a Clostridium botulinum, Clostridium butyricum, or Clostridium beratti bacterium. Additionally, the neurotoxin can be a modified neurotoxin; that is a neurotoxin that has at least one of its amino acids deleted, modified or replaced, as compared to the native or wild type neurotoxin. Furthermore, the neurotoxin can be a recombinantly produced neurotoxin or a derivative or fragment thereof.

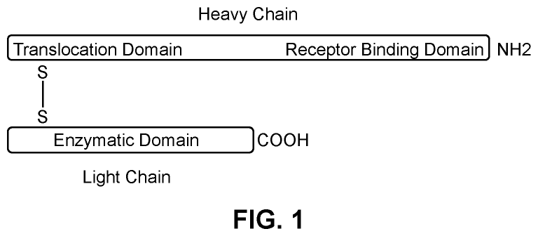

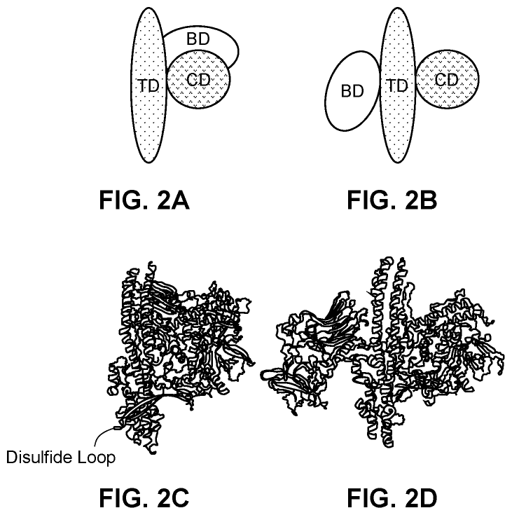

FIG. 1 depicts the primary structure of a botulinum neurotoxin (BoNT). FIGS. 2A-2D are schematic and ribbon representations of BoNT/E (FIGS. 2A, 2C) and BoNT/B (FIGS. 2B, 2D). The catalytic, translocation, and binding domains are labeled CD, TD, and BD, respectively.

In some embodiments, a disclosed botulinum neurotoxin type E (BoNT/E) composition has 40% amino acid homology compared with type A and they share the same basic domain structure consisting of 2 chains, a 100 kDa heavy chain (HC) and a 50 kDa light chain (LC), linked by a disulfide bond, see e.g. Whelan et al., The complete amino acid sequence of the Clostridium botulinum type-E neurotoxin, derived by nucleotide-sequence analysis of the encoding gene, Eur. J. Biochem. 204(2): 657-667 (1992). The HC contains the receptor binding domain and the translocation domain while the LC contains the synaptosomal-associated protein (SNAP) enzymatic activity. The domain structure is the same structure shared by all botulinum neurotoxin serotypes.

In disclosed embodiments, the neurotoxin is formulated in unit dosage form; for example, it can be provided as a sterile solution in a vial or as a vial or sachet containing a lyophilized powder for reconstituting a suitable vehicle such as saline for injection.

In some embodiments, the botulinum toxin is formulated in a solution containing saline and pasteurized human serum albumin, which stabilizes the toxin and minimizes loss through non-specific adsorption. The solution can be sterile filtered (0.2 μm filter), filled into individual vials and then vacuum-dried to give a sterile lyophilized powder. In use, the powder can be reconstituted by the addition of sterile unpreserved normal saline (sodium chloride 0.9% for injection).

In an embodiment, botulinum type E is supplied in a sterile solution for injection with a 5-mL vial nominal concentration of 20 ng/mL in 0.03 M sodium phosphate, 0.12 M sodium chloride, and 1 mg/mL Human Serum Albumin (HSA), at pH 6.0. In another embodiment, the botulinum type E is provided as a liquid formulation comprised of sodium phosphate, sodium chloride, and HSA. In yet another embodiment, the botulinum type E is supplied as a lyophilized or liquid formulation comprising a surfactant and a disaccharide.

Although the composition may only contain a single type of neurotoxin, for example botulinum type E, disclosed compositions can include two or more types of neurotoxins, which can provide enhanced therapeutic effects of the disorders. For example, a composition administered to a patient can include botulinum types A and E. Administering a single composition containing two different neurotoxins can permit the effective concentration of each of the neurotoxins to be lower than if a single neurotoxin is administered to the patient while still achieving the desired therapeutic effects. The composition administered to the patient can also contain other pharmaceutically active ingredients, such as, protein receptor or ion channel modulators, in combination with the neurotoxin or neurotoxins. These modulators may contribute to the reduction in neurotransmission between the various neurons. For example, a composition may contain gamma aminobutyric acid (GABA) type A receptor modulators that enhance the inhibitory effects mediated by the GABAA receptor. The GABAA receptor inhibits neuronal activity by effectively shunting current flow across the cell membrane. GABAA receptor modulators may enhance the inhibitory effects of the GABAA receptor and reduce electrical or chemical signal transmission from the neurons. Examples of GABAA receptor modulators include benzodiazepines, such as diazepam, oxaxepam, lorazepam, prazepam, alprazolam, halazeapam, chordiazepoxide, and chlorazepate. Compositions may also contain glutamate receptor modulators that decrease the excitatory effects mediated by glutamate receptors. Examples of glutamate receptor modulators include agents that inhibit current flux through AMPA, NMDA, and/or kainate types of glutamate receptors.

### Methods of Treatment

Methods disclosed herein can comprise administration of a fast-acting neurotoxin to a patient. In a preferred embodiment the neurotoxin is botulinum type E.

Methods disclosed herein can comprise supplemental administration of a fast-acting neurotoxin to a patient. Embodiments comprising supplemental administration can further comprise doctor or patient evaluation of the results of a prior neurotoxin administration.

In some embodiments, administration of the fast-acting neurotoxin is performed prior to a surgical procedure. In some embodiments, the administration is performed, for example, within 6 hours before the procedure, within 5 hours before the procedure, within 4 hours before the procedure, within 3 hours before the procedure, within 2 hours before the procedure, within 60 minutes before the procedure, within 50 minutes before the procedure, within 40 minutes before the procedure, within 30 minutes before the procedure, within 20 minutes before the procedure, within 10 minutes before the procedure, within 5 minutes before the procedure, within 2 minutes before the procedure, or the like.

In alternative embodiments, administration of the fast-acting neurotoxin is performed concurrently with a surgical procedure.

In other embodiments, administration of the fast-acting neurotoxin is performed after a surgical procedure. For example, administration can be performed, within 1 minute after the procedure, within 2 minutes after the procedure, within 3 minutes after the procedure, within 4 minutes after the procedure, within 5 minutes after the procedure, within 6 minutes after the procedure, within 7 minutes after the procedure, within 8 minutes after the procedure, within 9 minutes after the procedure, within 10 minutes after the procedure, within 20 minutes after the procedure, within 30 minutes after the procedure, within 40 minutes after the procedure, within 50 minutes after the procedure, within 60 minutes after the procedure, within 90 minutes after the procedure, within 120 minutes after the procedure, within 180 minutes after the procedure, within 240 minutes after the procedure, within 300 minutes after the procedure, or the like. In some other embodiments, administration can be performed within 1 to 3 days after the procedure. In yet other embodiments, administration can be performed within 3 months after the procedure.

In embodiments comprising a supplemental administration, evaluation of the results of the initial neurotoxin administration can be performed within, for example, 6 hours of the initial administration, 8 hours of the initial administration, 10 hours of the initial administration, 12 hours of the initial administration, 14 hours of the initial administration, 16 hours of the initial administration, 18 hours of the initial administration, 24 hours of the initial administration, 30 hours of the initial administration, 36 hours of the initial administration, 42 hours of the initial administration, 48 hours of the initial administration, 54 hours of the initial administration, 60 hours of the initial administration, 66 hours of the initial administration, 72 hours of the initial administration, 78 hours of the initial administration, 84 hours of the initial administration, 90 hours of the initial administration, 96 hours of the initial administration, 102 hours of the initial administration, 108 hours of the initial administration, 114 hours of the initial administration, 120 hours of the initial administration, 1 week of the initial administration, 2 weeks of the initial administration, 3 weeks of the initial administration, 4 weeks of the initial administration, 5 weeks of the initial administration, 6 weeks of the initial administration, 7 weeks of the initial administration, 8 weeks of the initial administration, 9 weeks of the initial administration, 10 weeks of the initial administration, 11 weeks of the initial administration, 12 weeks of the initial administration, or the like.

In embodiments comprising a supplemental administration, administration of the supplemental dose can be performed, within, for example, 6 hours of the evaluation, 8 hours of the evaluation, 10 hours of the evaluation, 12 hours of the evaluation, 14 hours of the evaluation, 16 hours of the evaluation, 18 hours of the evaluation, 24 hours of the evaluation, 30 hours of the evaluation, 36 hours of the evaluation, 42 hours of the evaluation, 48 hours of the evaluation, 54 hours of the evaluation, 60 hours of the evaluation, 66 hours of the evaluation, 72 hours of the evaluation, 78 hours of the evaluation, 84 hours of the evaluation, 90 hours of the evaluation, 96 hours of the evaluation, 102 hours of the evaluation, 108 hours of the evaluation, 114 hours of the evaluation, 120 hours of the evaluation, 1 week of the evaluation, 2 weeks of the evaluation, 3 weeks of the evaluation, 4 weeks of the evaluation, 5 weeks of the evaluation, 6 weeks of the evaluation, 7 weeks of the evaluation, 8 weeks of the evaluation, 9 weeks of the evaluation, 10 weeks of the evaluation, 11 weeks of the evaluation, 12 weeks of the evaluation, or the like.

In some embodiments, the supplemental administration itself can be performed, for example, within, for example, 6 hours of the initial administration of the fast-acting neurotoxin, 8 hours of the initial administration, 10 hours of the initial administration, 12 hours of the initial administration, 14 hours of the initial administration, 16 hours of the initial administration, 18 hours of the initial administration, 24 hours of the initial administration, 30 hours of the initial administration, 36 hours of the initial administration, 42 hours of the initial administration, 48 hours of the initial administration, 54 hours of the initial administration, 60 hours of the initial administration, 66 hours of the initial administration, 72 hours of the initial administration, 78 hours of the initial administration, 84 hours of the initial administration, 90 hours of the initial administration, 96 hours of the initial administration, 102 hours of the initial administration, 108 hours of the initial administration, 114 hours of the initial administration, 120 hours of the initial administration, 1 week of the initial administration, 2 weeks of the initial administration, 3 weeks of the initial administration, 4 weeks of the initial administration, 5 weeks of the initial administration, 6 weeks of the initial administration, 7 weeks of the initial administration, 8 weeks of the initial administration, 9 weeks of the initial administration, 10 weeks of the initial administration, 11 weeks of the initial administration, 12 weeks of the initial administration, or the like. In some embodiments, the supplemental administration can be performed, for example, within, for example, 3 to 6 months of the initial administration. In some embodiments, the supplemental administration can be performed concurrently as the initial administration.

Methods disclosed herein can provide rapid-onset effects (for example, using a fast-acting neurotoxin). For example, disclosed embodiments can reduce muscle activity in the proximity of a surgical incision within, for example, 30 minutes after administration, 45 minutes after administration, 60 minutes after administration, 75 minutes after administration, 90 minutes after administration, 2 hours after administration, 3 hours after administration, 4 hours after administration, 5 hours after administration, 6 hours after administration, 7 hours after administration, 8 hours after administration, 9 hours after administration, 10 hours after administration, 11 hours after administration, 12 hours after administration, 13 hours after administration, 14 hours after administration, 15 hours after administration, 16 hours after administration, 17 hours after administration, 18 hours after administration, 19 hours after administration, 20 hours after administration, 21 hours after administration, 22 hours after administration, 23 hours after administration, 24 hours after administration, 30 hours after administration, 36 hours after administration, 42 hours after administration, 48 hours after administration, 3 days after administration, 4 days after administration, 5 days after administration, 6 days after administration, 7 days after administration, or the like.

Methods disclosed herein can provide reduction in muscle activity for a shorter duration (for example, using a fast-recovery neurotoxin). For example, disclosed embodiments can provide a reduction in muscle activity that subsides within, for example, 3 days after administration, 4 days after administration, 5 days after administration, 6 days after administration, 7 days after administration, 8 days after administration, 9 days after administration, 10 days after administration, 11 days after administration, 12 days after administration, 13 days after administration, 14 days after administration, 15 days after administration, 16 days after administration, 17 days after administration, 18 days after administration, 19 days after administration, 20 days after administration, 21 days after administration, 22 days after administration, 23 days after administration, 24 days after administration, 25 days after administration, 26 days after administration, 27 days after administration, 28 days after administration, 29 days after administration, 30 days after administration, 45 days after administration, 60 days after administration, 75 days after administration, 90 days after administration, 105 days after administration, or the like.

Side-effects can be associated with botulinum injections. Disclosed embodiments can provide neurotoxin treatments that result in fewer side effects, or side effects of a shortened duration, than conventional neurotoxin treatments, for example treatment using a longer acting neurotoxin such as type A.

For example, disclosed embodiments can result in fewer (or shorter duration) instances of double vision or blurred vision, eyelid paralysis (subject cannot lift eyelid all the way open), loss of facial muscle movement, hoarseness, loss of bladder control, shortness of breath, difficulty in swallowing, difficulty speaking, death, and the like.

The disclosed methods comprise administration to an area prone to scarring, for example an area in the proximity of a surgical incision, or an area in the proximity of any injury to the skin, for example a traumatic injury. Disclosed embodiments comprise administration to muscles proximate to an area prone to scarring, for example, to skeletal muscle tissue or smooth muscle tissue.

Further, disclosed embodiments can provide reduced muscle activity of a more-certain duration. For example, with a longer acting neurotoxin, a 20% variance in duration of effects can result in a month's difference in effective duration. With the disclosed fast-recovery neurotoxins, this 20% variance produces a much less drastic difference in effective duration.

Disclosed fast-acting neurotoxin compositions can be injected into the individual using a needle or a needleless device. In certain embodiments, the method comprises subdermally injecting the composition in the individual. For example, the administering may comprise injecting the composition through a needle no greater than about 30 gauge. In certain embodiments, the method comprises administering a composition comprising a botulinum toxin type E.

Injection of the compositions can be carried out by syringe, catheters, needles and other means for injecting. The injection can be performed on any area of the mammal's body that is in need of treatment, including, but not limited to, face, neck, torso, arms, hands, legs, and feet. The injection can be into any position in the specific area such as epidermis, dermis, fat, muscle, or subcutaneous layer.

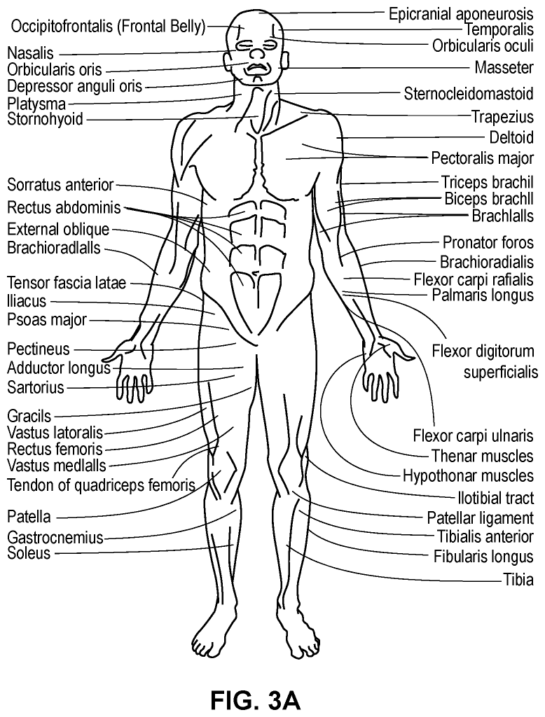

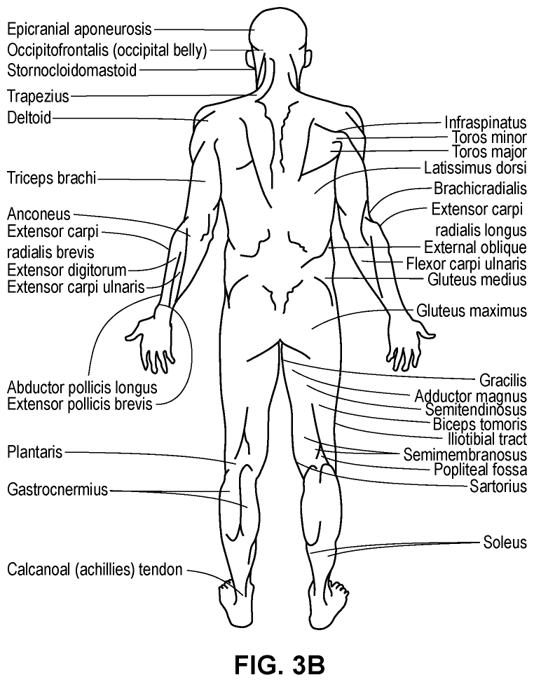

For example, skeletal muscles suitable for administration of disclosed compositions can comprise any of the muscles, or combinations of muscles, of the illustrations shown in FIGS. 3A-3B.

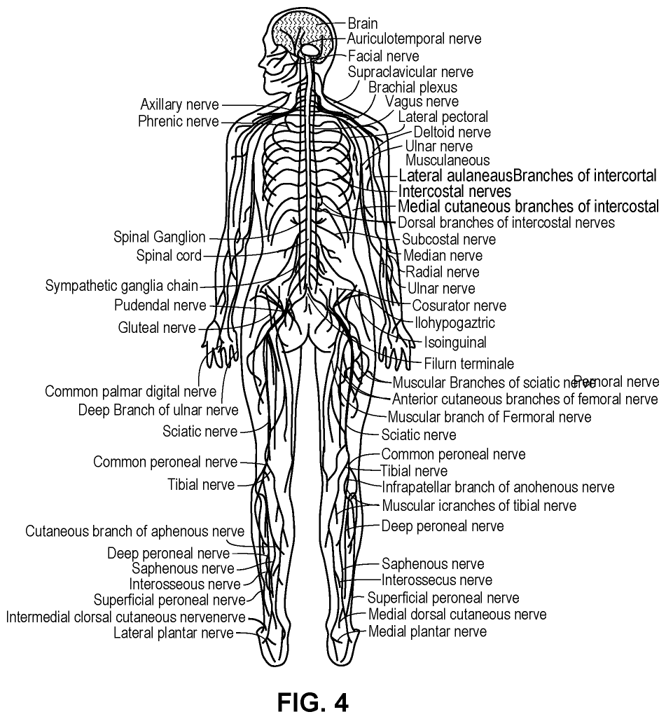

Administration can comprise injection into or in the vicinity of one or more of the nerves shown in the schematic of FIG. 4.

Smooth muscles suitable for administration of disclosed compositions can comprise any of walls of blood vessels, walls of stomach, ureters, intestines, in the aorta (tunica media layer), iris of the eye, prostate, gastrointestinal tract, respiratory tract, small arteries, arterioles, reproductive tracts (both genders), veins, glomeruli of the kidneys (called mesangial cells), bladder, uterus, arrector pili of the skin, ciliary muscle, sphincter, trachea, bile ducts, and the like.

The frequency and the amount of injection under the disclosed methods can be determined based on the nature and location of the particular area being treated. In certain cases, however, repeated injection may be desired to achieve optimal results. The frequency and the amount of the injection for each particular case can be determined by the person of ordinary skill in the art.

Although examples of routes of administration and dosages are provided, the appropriate route of administration and dosage are generally determined on a case by case basis by the attending physician. Such determinations are routine to one of ordinary skill in the art (see for example, Harrison's Principles of Internal Medicine (1998), edited by Anthony Fauci et al., 14th edition, published by McGraw Hill). For example, the route and dosage for administration of a Clostridial neurotoxin according to the present disclosed invention can be selected based upon criteria such as the solubility characteristics of the neurotoxin chosen as well as the intensity and scope of the condition being treated.

The fast-acting neurotoxin can be administered in an amount of between about 10⁻³ U/kg and about 35 U/kg. In an embodiment, the neurotoxin is administered in an amount of between about 10⁻² U/kg and about 25 U/kg. In another embodiment, the neurotoxin is administered in an amount of between about 10¹ U/kg and about 15 U/kg. In another embodiment, the neurotoxin is administered in an amount of between about 1 U/kg and about 10 U/kg. In many instances, an administration of from about 1 unit to about 500 units of a neurotoxin, such as a botulinum type E, provides effective therapeutic relief. In an embodiment, from about 5 units to about 200 units of a neurotoxin, such as a botulinum type E, can be used and in another embodiment, from about 10 units to about 100 units of a neurotoxin, such as a botulinum type E, can be locally administered into a target tissue such as a muscle.

In some embodiments, administration can comprise a dose of about 10 units of a neurotoxin, or about 20 units of a neurotoxin, or about 30 units of a neurotoxin, or about 40 units of a neurotoxin, or about 50 units of a neurotoxin, or about 60 units of a neurotoxin, or about 70 units of a neurotoxin, or about 80 units of a neurotoxin, or about 90 units of a neurotoxin, or about 100 units of a neurotoxin, or about 110 units of a neurotoxin, or about 120 units of a neurotoxin, or about 130 units of a neurotoxin, or about 140 units of a neurotoxin, or about 150 units of a neurotoxin, or about 160 units of a neurotoxin, or about 170 units of a neurotoxin, or about 180 units of a neurotoxin, or about 190 units of a neurotoxin, or about 200 units of a neurotoxin, or about 210 units of a neurotoxin, or about 220 units of a neurotoxin, or about 230 units of a neurotoxin, or about 240 units of a neurotoxin, or about 250 units of a neurotoxin, or about 260 units of a neurotoxin, or about 270 units of a neurotoxin, or about 280 units of a neurotoxin, or about 290 units of a neurotoxin, or about 290 units of a neurotoxin, or about 300 units of a neurotoxin, or about 310 units of a neurotoxin, or about 320 units of a neurotoxin, or about 330 units of a neurotoxin, or about 340 units of a neurotoxin, or about 350 units of a neurotoxin, or about 360 units of a neurotoxin, or about 370 units of a neurotoxin, or about 380 units of a neurotoxin, or about 390 units of a neurotoxin, or about 400 units of a neurotoxin, or about 410 units of a neurotoxin, or about 420 units of a neurotoxin, or about 430 units of a neurotoxin, or about 440 units of a neurotoxin, or about 450 units of a neurotoxin, or about 460 units of a neurotoxin, or about 470 units of a neurotoxin, or about 480 units of a neurotoxin, or about 490 units of a neurotoxin, or about 500 units of a neurotoxin, or the like.

In some embodiments, administration can comprise a dose of about 0.1 nanograms (ng) of a neurotoxin, 0.2 ng of a neurotoxin, 0.3 ng of a neurotoxin, 0.4 ng of a neurotoxin, 0.5 ng of a neurotoxin, 0.6 ng of a neurotoxin, 0.7 ng of a neurotoxin, 0.8 ng of a neurotoxin, 0.9 ng of a neurotoxin, 1.0 ng of a neurotoxin, 1.1 ng of a neurotoxin, 1.2 ng of a neurotoxin, 1.3 ng of a neurotoxin, 1.4 ng of a neurotoxin, 1.5 ng of a neurotoxin, 1.6 ng of a neurotoxin, 1.7 ng of a neurotoxin, 1.8 ng of a neurotoxin, 1.9 ng of a neurotoxin, 2.0 ng of a neurotoxin, 2.1 ng of a neurotoxin, 2.2 ng of a neurotoxin, 2.3 ng of a neurotoxin, 2.4 ng of a neurotoxin, 2.5 ng of a neurotoxin, 2.6 ng of a neurotoxin, 2.7 ng of a neurotoxin, 2.8 ng of a neurotoxin, 2.9 ng of a neurotoxin, 3.0 ng of a neurotoxin, 3.1 ng of a neurotoxin, 3.2 ng of a neurotoxin, 3.3 n of a neurotoxin, 3.4 ng of a neurotoxin, 3.5 ng of a neurotoxin, 3.6 ng of a neurotoxin, 3.7 ng of a neurotoxin, 3.8 ng of a neurotoxin, 3.9 ng of a neurotoxin, 4.0 ng of a neurotoxin, 4.1 ng of a neurotoxin, 4.2 ng of a neurotoxin, 4.3 ng of a neurotoxin, 4.4 ng of a neurotoxin, 4.5 ng of a neurotoxin, 5 ng of a neurotoxin, 6 ng of a neurotoxin, 7 ng of a neurotoxin, 8 ng of a neurotoxin, 9 ng of a neurotoxin, 10 ng of a neurotoxin, or the like.

In some embodiments, administration can comprise a dose of between about 0.1 nanograms (ng) of a neurotoxin and 20 ng of a neurotoxin, between about 1 ng of a neurotoxin and 19 ng of a neurotoxin, between about 2 ng of a neurotoxin and 18 ng of a neurotoxin, between about 3 ng of a neurotoxin and 17 ng of a neurotoxin, between about 4 ng of a neurotoxin and 16 ng of a neurotoxin, between about 5 ng of a neurotoxin and 15 ng of a neurotoxin, between about 6 ng of a neurotoxin and 14 ng of a neurotoxin, between about 7 ng of a neurotoxin and 13 ng of a neurotoxin, between about 8 ng of a neurotoxin and 12 ng of a neurotoxin, between about 9 ng of a neurotoxin and 11 ng of a neurotoxin, or the like. In embodiments, administration can comprise one or more injections of about 0.1 nanograms (ng) of a neurotoxin, 0.2 ng of a neurotoxin, 0.3 ng of a neurotoxin, 0.4 ng of a neurotoxin, 0.5 ng of a neurotoxin, 0.6 ng of a neurotoxin, 0.7 ng of a neurotoxin, 0.8 ng of a neurotoxin, 0.9 ng of a neurotoxin, 1.0 ng of a neurotoxin, 1.1 ng of a neurotoxin, 1.2 ng of a neurotoxin, 1.3 ng of a neurotoxin, 1.4 ng of a neurotoxin, 1.5 ng of a neurotoxin, 1.6 ng of a neurotoxin, 1.7 ng of a neurotoxin, 1.8 ng of a neurotoxin, 1.9 ng of a neurotoxin, 2.0 ng of a neurotoxin, 2.1 ng of a neurotoxin, 2.2 ng of a neurotoxin, 2.3 ng of a neurotoxin, 2.4 ng of a neurotoxin, 2.5 ng of a neurotoxin, 2.6 ng of a neurotoxin, 2.7 ng of a neurotoxin, 2.8 ng of a neurotoxin, 2.9 ng of a neurotoxin, 3.0 ng of a neurotoxin, 3.1 ng of a neurotoxin, 3.2 ng of a neurotoxin, 3.3 n of a neurotoxin, 3.4 ng of a neurotoxin, 3.5 ng of a neurotoxin, 3.6 ng of a neurotoxin, 3.7 ng of a neurotoxin, 3.8 ng of a neurotoxin, 3.9 ng of a neurotoxin, 4.0 ng of a neurotoxin, 4.1 ng of a neurotoxin, 4.2 ng of a neurotoxin, 4.3 ng of a neurotoxin, 4.4 ng of a neurotoxin, 4.5 ng of a neurotoxin, 5 ng of a neurotoxin, 6 ng of a neurotoxin, 7 ng of a neurotoxin, 8 ng of a neurotoxin, 9 ng of a neurotoxin, 10 ng of a neurotoxin, or the like.

In some embodiments, administration can comprise one or more injections of between about 0.1 nanograms (ng) of a neurotoxin and 20 ng of a neurotoxin, between about 1 ng of a neurotoxin and 19 ng of a neurotoxin, between about 2 ng of a neurotoxin and 18 ng of a neurotoxin, between about 3 ng of a neurotoxin and 17 ng of a neurotoxin, between about 4 ng of a neurotoxin and 16 ng of a neurotoxin, between about 5 ng of a neurotoxin and 15 ng of a neurotoxin, between about 6 ng of a neurotoxin and 14 ng of a neurotoxin, between about 7 ng of a neurotoxin and 13 ng of a neurotoxin, between about 8 ng of a neurotoxin and 12 ng of a neurotoxin, between about 9 ng of a neurotoxin and 11 ng of a neurotoxin, or the like.

Ultimately, however, both the quantity of toxin administered and the frequency of its administration will be at the discretion of the physician responsible for the treatment and will be commensurate with questions of safety and the effects produced by the toxin.

In some embodiments, administration can comprise one or more injections, for example injections substantially along the incision site or line or lines. In some embodiments, administration can comprise injections in a specific pattern, for example, a W pattern, and X patter, a Z pattern, a star pattern, a circle pattern, a half circle pattern, a square pattern, a rectangle pattern, a crescent patter, or combinations thereof.

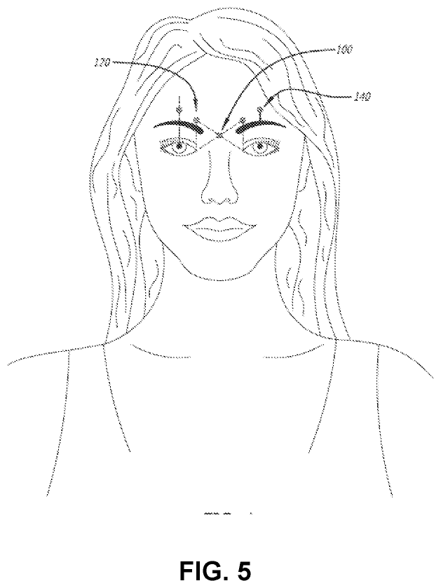

A study was conducted where BoNT/E was administered to human patients undergoing treatment of glabellar lines, in order to evaluate onset of action, safety and tolerability of BoNT/E in humans. The study is described in Example 1. The BoNT/E was administered into the procerus and corrugator muscles at five injection points, as illustrated in FIG. 5. BoNT/E provided improvement in severity of glabellar lines with fast onset of action, and with a favorable safety and tolerability profile.

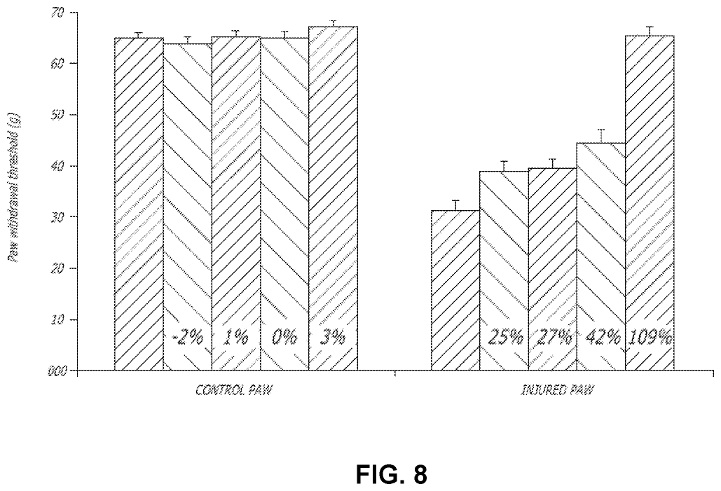

Another study was conducted where BoNT/E was administered to rats in an animal model of Post-Operative Pain in order to evaluate onset of action and efficacy of BoNT/E in reducing post-operative pain. As described in Example 9, in anestherized rats, a 1 cm longitudinal incision was made through the skin, fascia and muscle of the plantar aspect of the hindpaw. The uninjured paw was used as a control paw. Twenty-four hours after surgery, the incision produced a mechanical allodynia which was quantified using the electronic Von Fret test. Three doses of BoNT/E were administered into the hindpaw of both the injured paw and control paw 24 hours prior to surgery. The pain threshold was evaluated with electron Von Frey test 24 hours post surgery. In one group, morphine was used to assess the maximum pain reduction achievable. The results are shown in FIG. 8 and expressed as increase (+) or decrease (−) as compared to the vehicle-treated group. BoNT/E provided significant increase in pain threshold which demonstrate the pain reduction ability of BoNT/E, as shown in FIG. 8.

A controlled release system can be used in the embodiments described herein to deliver a neurotoxin in vivo at a predetermined rate over a specific time period. Generally, release rates are determined by the design of the system and can be largely independent of environmental conditions such as pH. Controlled release systems which can deliver a drug over a period of several years are known. Contrarily, sustained release systems typically deliver drug in 24 hours or less and environmental factors can influence the release rate. Thus, the release rate of a neurotoxin from an implanted controlled release system (an “implant”) is a function of the physiochemical properties of the carrier implant material and of the drug itself. Typically, the implant is made of an inert material which elicits little or no host response.

A controlled release system can be comprised of a neurotoxin incorporated into a carrier. The carrier can be a polymer or a bio-ceramic material. The controlled release system can be injected, inserted or implanted into a selected location of a patient's body and reside therein for a prolonged period during which the neurotoxin is released by the implant in a manner and at a concentration which provides a desired therapeutic efficacy.

Polymeric materials can release neurotoxins due to diffusion, chemical reaction or solvent activation, as well as upon influence by magnetic, ultrasound or temperature change factors. Diffusion can be from a reservoir or matrix. Chemical control can be due to polymer degradation or cleavage of the drug from the polymer. Solvent activation can involve swelling of the polymer or an osmotic effect.

Implants may be prepared by mixing a desired amount of a stabilized neurotoxin into a solution of a suitable polymer dissolved in methylene chloride. The solution may be prepared at room temperature. The solution can then be transferred to a Petri dish and the methylene chloride evaporated in a vacuum desiccator. Depending upon the implant size desired and hence the amount of incorporated neurotoxin, a suitable amount of the dried neurotoxin incorporating implant is compressed at about 8000 p.s.i. for 5 seconds or at 3000 p.s.i. for 17 seconds in a mold to form implant discs encapsulating the neurotoxin.

Preferably, the implant material used is substantially non-toxic, non-carcinogenic, and non-immunogenic. Suitable implant materials include polymers, such as poly(2-hydroxy ethyl methacrylate) (p-HEMA), poly(N-vinyl pyrrolidone) (p-NVP)+, poly(vinyl alcohol) (PVA), poly(acrylic acid) (PM), polydimethyl siloxanes (PDMS), ethylene-vinyl acetate (EVAc) copolymers, polyvinylpyrrolidone/methylacrylate copolymers, polymethylmethacrylate (PMMA), poly(lactic acid) (PLA), poly(glycolic acid) (PGA), polyanhydrides, poly(ortho esters), collagen and cellulosic derivatives and bioceramics, such as hydroxyapatite (HPA), tricalcium phosphate (TCP), and aliminocalcium phosphate (ALCAP). Lactic acid, glycolic acid and collagen can be used to make biodegradable implants.

An implant material can be biodegradable or bioerodible. An advantage of a bioerodible implant is that it does not need to be removed from the patient. A bioerodible implant can be based upon either a membrane or matrix release of the bioactive substance. Biodegradable microspheres prepared from PLA-PGA are known for subcutaneous or intramuscular administration.

A kit for practicing disclosed embodiments is also encompassed by the present disclosure. The kit can comprise a 30 gauge or smaller needle and a corresponding syringe. The kit also comprises a Clostridial neurotoxin composition, such as a botulinum type E toxin composition. The neurotoxin composition may be provided in the syringe. The composition is injectable through the needle. The kits are designed in various forms based the sizes of the syringe and the needles and the volume of the injectable composition contained therein, which in turn are based on the specific deficiencies the kits are designed to treat.

### EXAMPLES

The following non-limiting examples are provided for illustrative purposes only in order to facilitate a more complete understanding of representative embodiments. This example should not be construed to limit any of the embodiments described in the present specification.

### Example 1

### Use of Botulinum Toxin Type E to Treat Glabellar Lines

A randomized, double-blind, placebo-controlled, ascending dose cohort, Phase 2a study in humans was conducted to evaluate safety and efficacy of a single treatment cycle of a disclosed fast-acting type E composition in subjects with glabellar frown lines. This study was conducted in compliance with this protocol and all applicable Federal and State regulations. A composition composed of botulinum neurotoxin subtype E (BoNT/E, “EB-001”) was administered to the subjects. This first-in-human, randomized, double-blinded, placebo-controlled, ascending dose cohort study enrolled 42 subjects who received EB-001 (a botulinum type E composition disclosed herein) (N=35) or placebo (N=7). The efficacy primary outcome was the proportion of subjects with a 2-grade investigator-rated (IR-2) improvement in GL severity at maximum frown. Safety evaluations included adverse events (AEs), laboratory tests, and physical examinations. An IR-2 response was observed starting in the third cohort (EB-001), with increased rates observed at higher doses. Onset of clinical effect was within 24 hours, with a duration ranging between 14 and 30 days for the highest doses. AE incidence was low, with the most common being mild to moderate headache. There were no serious AEs or ptosis, and no clinically significant changes in other safety assessments.

In this clinical study in GL, EB-001 showed favorable safety and tolerability, and dose dependent efficacy with an 80% response rate at the highest dose. EB-001 maximum clinical effect was seen within 24 hours and lasted between 14 and 30 days. This differentiated EB-001 profile supports its development for aesthetic and therapeutic applications where fast onset and short duration of effect are desirable.

Botulinum neurotoxins, which inhibit the pre-synaptic release of acetylcholine, are among the most potent molecules in nature. When injected into muscles, Botulinum neurotoxins inhibit neuromuscular transmission and produce dose-dependent local muscle relaxation. Purified Botulinum neurotoxins, including serotypes A and B have been developed as injectable drugs and are widely used to treat a variety of neuromuscular conditions. Botulinum neurotoxin serotype E is a novel serotype that has not been developed for clinical use to date. Botulinum toxin type E has the fastest onset and the shortest duration of action of all the Botulinum neurotoxins. Type E has similar domain structure to type A, consisting of 2 protein chains, a 100 kDa heavy chain and a 50 kDa light chain linked by a disulfide bond, as shown in FIG. 1. Type E inhibits neuromuscular transmission by cleaving the same presynaptic vesicular protein (synaptosomal associated protein 25) as type A, but at a different cleavage site. Two binding sites on motor axons mediate the high affinity recognition of nerve cells by Botulinum neurotoxins. Binding is mediated first by cell surface gangliosides and then by specific protein receptors. These receptors are found on motor axon terminals at the neuromuscular junction. Botulinum toxin types A and E have both been shown to bind the specific receptor synaptic vesicle protein 2, and only these two serotypes share this receptor. This was the first clinical study to evaluate the safety and efficacy of ascending doses of Botulinum toxin type E in subjects with GL.

This study was a first-in-human evaluation of the safety and efficacy of EB-001 and focused on the treatment of moderate to severe GL. EB-001 is a proprietary purified form of Botulinum toxin type E, formulated as a liquid for injection (Bonti, Inc., Newport Beach, Calif., USA). This was a randomized, double-blinded, placebo-controlled, ascending-dose cohort study conducted at 2 expert clinical centers (Steve Yoelin, MD Medical Associates, Newport Beach, Calif., USA; Center for Dermatology Clinical Research, Fremont, Calif., USA). This study was approved by an Institutional Review Board (Aspire Institutional Review Board, Santee, Calif., USA) and was conducted in accordance with the guidelines set by the Declaration of Helsinki. Written informed consent was received from all subjects prior to their participation.

A total of 42 healthy toxin-naïve male and female subjects, ages 18 to 60 years, were enrolled in the study. Each subject's participation was to last approximately 6 weeks. The main inclusion criteria were: the presence of bilaterally symmetrical GL of moderate to severe rating at maximum frown, sufficient visual acuity without the use of eyeglasses (contact lens use acceptable) to accurately assess their facial wrinkles, and the ability to conform with study requirements. The main criteria for exclusion were: any uncontrolled systemic disease or other medical condition, any medical condition that may have put the subject at increased risk with exposure to Botulinum neurotoxin (including diagnosed myasthenia gravis, Eaton-Lambert syndrome, amyotrophic lateral sclerosis, or any other condition that interfered with neuromuscular function), current or prior Botulinum neurotoxin treatment, known immunization or hypersensitivity to Botulinum neurotoxin, pre-specified dermatological procedures within 3 to 12 months of the study (non-ablative resurfacing, facial cosmetic procedures, topical/oral retinoid therapy, etc.), and prior periorbital surgery or treatment. Women were not enrolled if they were pregnant, lactating, or planning to become pregnant. Men with female partner(s) of childbearing potential were enrolled only if they agreed to use dual methods of contraception for 3 months following dosing.

At Screening, subject demographics, medical history, and prior and concomitant medications were recorded and an alcohol/drug screen was performed. Standardized facial photography was performed at Baseline prior to treatment, and at every follow-up visit through the end of the study, but the photographs were not used for efficacy evaluations.

Seven cohorts (6 subjects per cohort) were enrolled and received ascending doses of EB-001 or placebo in a 5:1 ratio. The maximum recommended starting dose (with a 10-fold safety factor) in this first-in-human study was developed based on the no observed adverse effect levels from a preclinical safety and toxicity study (unpublished data). From this, a base dose (Cohort 1) was calculated and determined to be sub-efficacious, and Cohorts 2 to 7 received 3, 9, 12, 16, 21, and 28 times the base dose, respectively. This represented sub-efficacious to maximum-efficacious doses of EB-001. The total dose was delivered at 5 injection sites in equal volumes (0.1 mL per site into the procerus, left and right medial corrugators, and left and right lateral corrugators) in a standardized fashion (see FIG. 5). The spacing of injections into the lateral corrugators was approximately 1 cm above the supraorbital ridge. EB-001 was supplied in a sterile solution for injection in a 5-mL vial. The placebo was supplied in identical vials without EB-001.

Each subject completed visits at Screening (Day −30 to −1), Baseline/Injection (Day 0), Days 1, 2, 7, 14, and 30 (end of study), and Day 42 (final safety follow-up).

Safety was evaluated by adverse events (AEs), laboratory testing, electrocardiograms (ECGs), physical examinations, vital signs (pulse rate, respiratory rate, and blood pressure), urine pregnancy tests (for women of childbearing potential), and focused neurologic examinations to evaluate for the potential spread of Botulinum neurotoxin. Treatment-emergent AEs (TEAEs) were defined as any AE that started or worsened in severity after exposure to study treatment. AEs and TEAEs were summarized by system organ class and preferred term using the Medical Dictionary for Regulatory Activities (MedDRA, version 19.0). Serious AEs (SAEs, or AEs that fulfilled regulatory criteria for medical seriousness), and discontinuation due to AEs were also evaluated. Severity of AEs was recorded as mild, moderate, severe, or life threatening. Before enrollment of each dosing cohort, a safety data review committee met to analyze all safety data from the previous cohort(s).

At Screening, Baseline, and Days 1, 2, 7, 14, and 30, the subject's GL were assessed at maximum frown and at rest using the Facial Wrinkle Scale (FWS). Evaluations were completed by the investigator and the subject. The FWS is a widely accepted measure used for the evaluation of facial line severity. In the present study, the 4-point scale indicating severity of GL was as follows: 0=none, 1=mild, 2=moderate, 3=severe. Subjects were considered as treatment responders if they achieved at least a 2-grade improvement (reduction) based on the investigator's FWS assessment (IR-2). The primary efficacy variable was the proportion of IR-2 responders at maximum frown at any post baseline visit through Day 30. An additional efficacy endpoint of interest was the proportion of responders achieving an investigator-assessed FWS grade of none or mild at Days 1, 2, 7, 14, or 30 (analyzed by visit).

Two analysis populations were pre-specified, a safety and an efficacy population. Subjects receiving placebo were pooled for all analyses. The safety population included all subjects who received study treatment and had at least 1 safety assessment thereafter. All TEAEs and SAEs were summarized by treatment group. All safety parameters, including laboratory testing, ECGs, physical exams, vital signs, urine pregnancy tests, and focused neurologic examinations, were reviewed and evaluated for clinical significance by the investigators. The efficacy population was the modified intent-to-treat (mITT) population, defined as all randomized subjects who received at least 1 dose of study treatment and had at least 1 post baseline efficacy assessment. Analyses of demographics and baseline characteristics were performed on the mITT population. Medical history was based on the safety population and coded using MedDRA and summarized by system organ class and preferred term. Prior and concomitant medications were based on the safety population and coded using the World Health Organization Anatomical Therapeutic Chemical classification index and summarized by drug class and treatment group. Efficacy analyses were performed using the mITT population. FWS grades were summarized by treatment and study day using frequency counts and rates of response (%). An analysis comparing the proportion of IR-2 responders in each EB-001 cohort versus placebo (pooled) was performed using Fisher's exact test with a 0.05 level of significance.

Of the 59 subjects who were screened for the study, 43 were enrolled into 1 of 7 cohorts. One subject did not receive treatment, and consequently 42 subjects were included in the mITT and safety populations (35 treated with EB-001 and 7 treated with placebo). Forty-one subjects completed the study, with 1 subject lost to follow-up. The mean (range) ages of subjects for the EB-001 (pooled) versus placebo (pooled) groups were 47.9 (22 to 60) and 50.4 (32 to 57) years, respectively. The majority of subjects were female (EB-001=91.4%; placebo=85.7%) and white (71.4% for both groups). The baseline mean (standard deviation [SD]) investigator-assessed GL at maximum frown were 2.6 (0.50) and 2.9 (0.38) for the EB-001 and placebo groups, respectively. The EB-001 and placebo groups were well balanced with no substantial between-group differences.

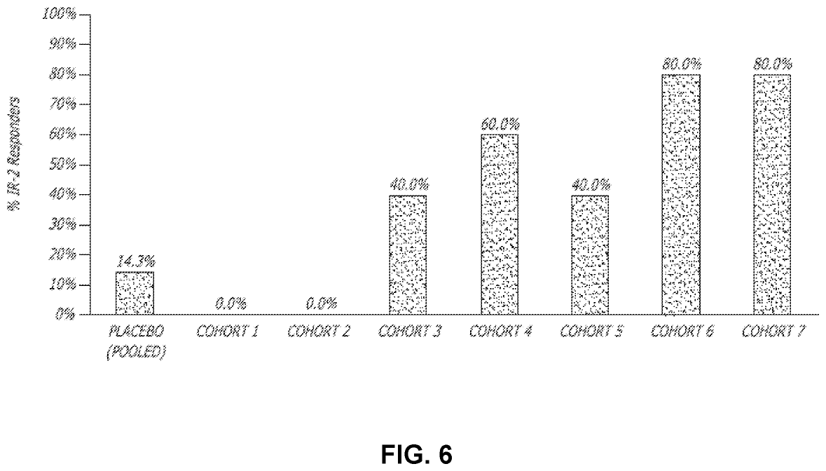

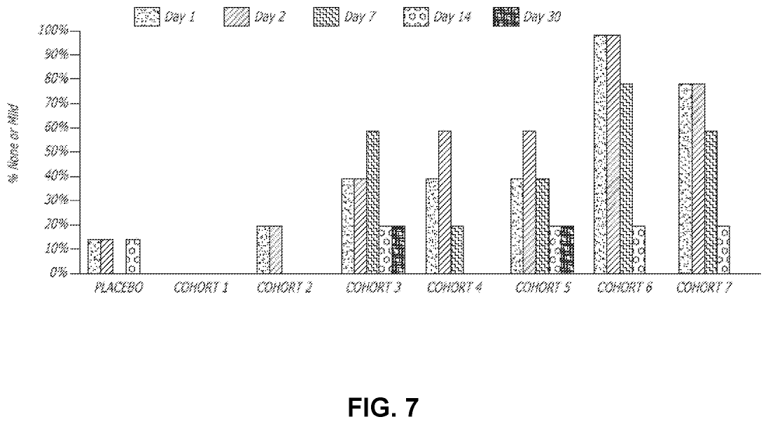

The proportions of subjects in the mITT population achieving an IR-2 response for GL severity at maximum frown at any postbaseline visit through Day 30 are presented by dose cohort in FIG. 6. In Cohort 3, 40% of subjects were IR-2 responders. This responder rate was the same or greater in all higher dose cohorts, with Cohorts 6 and 7 having 80% IR-2 responders. Cohorts 6 and 7 demonstrated significantly greater percentages of IR-2 responders versus placebo (P=0.046). FIG. 7 summarizes the proportions of subjects in each cohort with investigator-assessed FWS grades of none or mild GL at maximum frown, at any post baseline visit through Day 30. Cohorts 2 to 7 (inclusive) had greater percentages of responders versus placebo, with rates of 60% to 100% achieved for Cohorts 3 and higher. In Cohorts 3 to 7, most none or mild responses were observed at Days 1, 2, and/or 7. One responder (20%) was observed at Day 14 in Cohorts 3, 5, 6 and 7 and at Day 30 in Cohorts 3 and 5. The safety results support the safety of all evaluated doses of EB-001, administered as IM injections, in this population. No clinically significant changes from baseline in neurologic examinations, ECGs, physical examinations, or laboratory tests were observed for any subject.

Five subjects treated with EB-001 reported TEAEs, and none in placebo group. No SAEs were reported and no TEAE led to discontinuation of the study. All TEAEs were mild or moderate in severity. The events of sore throat and flu like symptoms were considered unrelated to treatment. Three subjects reported TEAEs of headache, 1 of which was considered related to treatment. There was no dose-related increase in the incidence of headaches. There were no events of ptosis or other TEAE possibly related to spread of toxin.

To our knowledge, this is the first controlled clinical trial of a Botulinum toxin type E product in any aesthetic or therapeutic use. This first-in-human study of EB-001, a novel purified form of Botulinum toxin type E administered IM, fulfilled its objectives of evaluating the safety, tolerability, and efficacious dose-range of EB-001. A dose response was observed, with greater proportions of treatment responders in the higher dosing cohorts of EB-001. An IR-2 response was observed starting with Cohort 3 and increased in higher dose cohorts, suggesting that the efficacious dose range of EB-001 may be at doses used in Cohorts 4 to 7. Cohorts 6 and 7 had 80% IR-2 responders, a response rate similar to approved Botulinum toxin type A products. Subjects achieving none or mild FWS grades were observed starting at Cohort 2. In terms of onset of effect, treatment response was observed as early as 24 hours following dosing, which supports prior reports suggesting that Botulinum toxin type E has a faster onset than type A.

Regarding the duration of effect defined as the proportion of responders with a none or mild rating, an effect was observed through Day 14 in 1 subject in most of the 5 higher dose cohorts, and through Day 30 in 1 subject in 2 of the 5 higher dose cohorts. All doses of EB-001 showed good tolerability with no local injection site reactions. There were no SAEs or severe TEAEs reported, and no discontinuations due to a TEAE. The most common TEAE of headache was mild or moderate in severity, and there were no other treatment related AEs. There were no events of ptosis at any dose levels, and no events potentially related to spread of toxin. Therefore, the clinical safety and tolerability profile seems favorable in this study. The efficacy and safety profiles of EB-001 are promising and support the potential of EB-001 as a unique treatment option in the treatment of GL and other facial aesthetic uses. The fast onset can fulfill an unmet need for individuals seeking a rapid treatment for facial wrinkles before unexpected social or professional events. The limited duration of effect can be beneficial for individuals who may be considering first time use of a Botulinum neurotoxin treatment, and are unwilling to make a longer-term commitment. An EB-001 treatment would allow them to assess the aesthetic effect over a shorter duration of effect compared with the 12-week duration of effect of Botulinum toxin type A products. In this first clinical study in subjects with GL, EB-001 showed favorable safety and tolerability in all cohorts. Five out of the 7 cohorts showed numerically higher response rates compared to placebo, supporting the efficacy of EB-001 in the reduction of GL severity. The 2 highest doses provided an 80% response rate, similar to approved Botulinum toxin type A products. In contrast to the known time course of type A products, the clinical effect of EB-001 was seen within 24 hours (onset) and lasted between 14-30 days (duration). This differentiated clinical profile supports the future development of EB-001 for facial aesthetic and key therapeutic uses, where fast onset and short duration of effect are desirable.

The total dose was distributed as follows:

| | Total | | | |
|---|---|---|---|---|
| | BoNT/E | Dose at | Doses at medial | Dose at lateral |
| | (EB-001) | Procerus | corrugators | corrugators |
| Cohort | Dose (ng) | (ng) | (ng) | (ng) |
| 1 | 0.1 | 0.02 | 0.02 into each of | 0.02 into each of |
| | | | right and left | right and left |
| | | | corrugators | corrugators |
| 2 | 0.3 | 0.06 | 0.06 into each of | 0.06 into each of |
| | | | right and left | right and left |
| | | | corrugators | corrugators |
| 3 | 0.9 | 0.18 | 0.18 into each of | 0.18 into each of |
| | | | right and left | right and left |
| | | | corrugators | corrugators |
| 4 | 1.2 | 0.24 | 0.24 into each of | 0.24 into each of |
| | | | right and left | right and left |
| | | | corrugators | corrugators |
| 5 | 1.6 | 0.32 | 0.32 into each of | 0.32 into each of |
| | | | right and left | right and left |
| | | | corrugators | corrugators |
| 6 | 2.1 | 0.42 | 0.42 into each of | 0.42 into each of |
| | | | right and left | right and left |
| | | | corrugators | corrugators |
| 7 | 2.8 | 0.56 | 0.56 into each of | 0.56 into each of |
| | | | right and left | right and left |
| | | | corrugators | corrugators |

### Example 2

### Use of Botulinum Toxin Type E to Treat Bacterial Necrotizing Lesion

A 60-year-old woman presents with a bacterial necrotizing legion on her leg. 12 hours prior to a debridement procedure, botulinum type E is injected around the perimeter of the lesion (6 equally-spaced injection sites; 5U injected to each site). The botulinum type E reduces muscle activity around the lesion as well as block neuronal release of CGRP. Within a week the lesion is healing.

### Example 3

### Use of Botulinum Toxin Type E to Treat Bacterial Necrotizing Lesion

A 30-year-old woman presents with a bacterial necrotizing legion on his arm. Immediately after diagnosis, botulinum type E is injected around the perimeter of the lesion (15 equally-spaced injection sites; 3U injected to each site). The botulinum type E reduces muscle activity around the lesion and blocks neuronal release of CGRP. Within a week the lesion is healing.

### Example 4

### Use of Botulinum Toxin Type E Treat Bacterial Necrotizing Lesion

A 14-year-old boy presents with a bacterial necrotizing legion on his arm. Immediately after diagnosis, botulinum type E is injected around the perimeter of the lesion (15 equally-spaced injection sites; 3U injected to each site). The botulinum type E reduces muscle activity around the lesion and blocks neuronal release of CGRP. Within a week the lesion is healing.

### Example 5

### Use of Botulinum Toxin Type E and Type A to Treat Bacterial Necrotizing Lesion

A 24-year-old woman presents with a bacterial necrotizing legion on her abdomen. Immediately after diagnosis, a combination of botulinum types E and A is injected around the perimeter of the lesion (12 equally-spaced injection sites; 4U injected to each site). The botulinum reduces muscle activity around the lesion and blocks neuronal release of CGRP. Within a week the lesion is healing.

### Example 5

### Use of Botulinum Toxin Type E and Type A to Treat Bacterial Necrotizing Lesion

A 24-year-old woman presents with a bacterial necrotizing legion on her abdomen. Immediately after diagnosis, a combination of botulinum types E and A is injected around the perimeter of the lesion (12 equally-spaced injection sites; 4U injected to each site). The botulinum reduces muscle activity around the lesion and blocks neuronal release of CGRP. Within a week the lesion is healing.

### Example 6

### Use of Botulinum Toxin Type E A to Treat Migraine Pain

A 40-year-old man suffering from chronic migraines is injected with a botulinum type E around the skull (20 equally-spaced injection sites; 2U injected to each site). The patient is migraine-free for the next month.

### Example 7

### Use of Botulinum Toxin Type E to Treat Burn Pain

A 14-year-old girl presents with a 2″ by 2″ third degree burn on her arm. Botulinum type E is administered around the perimeter of the burn, for a total of 10 injections of 1 unit each. Pain relief is felt within 48 hours and lasts for 4 weeks.

### Example 8

### Use of Botulinum Toxin Type E to Treat Inflammation

A 55-year-old male presents with an infection in his lower leg, accompanied by localized swelling. 20 injections of 0.5 ng per injection are administered to the patient's lower leg. Within 36 hours the inflammation decreases.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present disclosure, which is defined solely by the claims. Accordingly, embodiments of the present disclosure are not limited to those precisely as shown and described.

### Example 9

### Use of Botulinum Toxin Type E to Treat Post-Operative Pain

The effect of a single local administration of BoNT/E to treat post-operative pain was evaluated in a rat model of Post-operative pain, known as the Brennan model. Briefly, the model allows assessment of increased mechanical sensitivity following a surgical incision. In anesthetized rats, a 1-cm longitudinal incision was made through skin, fascia and muscle of the plantar aspect of the hindpaw. The control paw refers to the paw where there was no surgical injury. Twenty-four hours after surgery, the incision produced a mechanical allodynia which was quantified using the electronic Von Fret test. Three doses of BoNT/E were administered into the hindpaw of both the injured paw and control paw 24 hours prior to surgery. The pain threshold was evaluated with electron Von Frey test 24 hours post surgery. In one group, morphine was used to assess the maximum pain reduction achievable. The results are shown in FIG. 8 and expressed as increase (+) or decrease (−) as compared to the vehicle-treated group. Referring to FIG. 8, for both the control paw and the injured paw: (1) the first column shows data obtained for the vehicle-treated group, (2) the second, third and fourth columns show data obtained for the BoNT/E treated groups in increasing concentrations of 0.75 ng/kg, 1 ng/kg and 2 ng/kg, respectively; and (3) the last column shows data obtained in a morphine-treated group. In this Brennan rat model for post-operative pain, when a treatment is effective, the pain threshold goes back up closer to the value of the control uninjured paw. As shown in FIG. 8, the morphine was able to completely reduce the pain threshold to that of the control uninjured paw. The BoNT/E treated paws showed a dose dependent pain reduction.

The effect of BONT/E on muscle relaxation was also evaluated in a DAS model of muscle relaxation. Briefly, the DAS assay investigates the effects of BoNT preparations on the mouse hindlimb digit abduction (“startle”, “toe spread”) behavioral reflex in response to hindlimb elevation (tail-lift). The degree of paralysis is measured by scoring the ability of the mouse to abduct digits of the hindlimb paw. Loss of abduction is scored from zero to four, where four represents total loss of ability to abduct, and zero represents full ability to abduct the digits. The DAS assay was performed on the injured and uninjured paws treated with BoNT/E as described above. It was found (data not shown) that a dose response profile was observed with the BoNT/E treated groups, demonstrating muscle relaxation activity as measured by DAS response, wherein: (1) BoNT/E showed a dose-dependent effect in the DAS assay with doses of 0.75 ng/kg to 2 ng/kg; (2) onset of clinical effect was ˜12 hours, regardless of dose; (3) onset of maximal clinical effect was ˜24 hours, regardless of the dose; and (4) the duration of effect was longer at the highest dose ˜72 hours, and 48 hours at the two lower doses. The shorter duration of muscle weakness obtained with BoNT/E may allow faster recovery and rehabilitation post-operatively.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context. Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

1. A method for treating an infection, comprising:
administering a therapeutically effective amount of a fast-acting neurotoxin to a patient in the proximity of the infection.

2. The method of claim 1, wherein said fast-acting neurotoxin comprises botulinum neurotoxin serotype E.

3. The method of claim 2, wherein said therapeutically effective amount comprises an amount of between about 10⁻³ U/kg and about 35 U/kg.

4. The method of claim 4, wherein said therapeutically effective amount comprises an amount of between about 1 U/kg and about 25 U/kg.

5. The method of claim 4, wherein said therapeutically effective amount comprises an amount of between about 5 U/kg and about 15 U/kg.

6. The method of claim 2, wherein said therapeutically effective amount comprises an amount of between about 0.2 nanograms and about 2 nanograms.

7. The method of claim 6, wherein said therapeutically effective amount comprises an amount of between about 0.5 nanograms and about 1 nanogram.

8. The method of claim 2, wherein said administering comprises administering by injection.

9. The method of claim 8, wherein said administering by injection is an intramuscular injection.

10. The method of claim 2, wherein said method further comprises administering a botulinum toxin subtype A to the patient.

11. The method of claim 2, wherein said method further comprises administering onabotulinumtoxinA to the patient to the patient.

12. The method of claim 2, wherein said method further comprises administering a slower-recovery toxin.

13. The method of claim 2, wherein said method further comprises surgical debridement.

14. A method for inhibiting or reducing cGRP release to a patient in need thereof, the method comprising:
administering a therapeutically effective amount of a fast-acting neurotoxin to the patient.

15. The method of claim 14, wherein said fast-acting neurotoxin comprises botulinum neurotoxin serotype E (BoNT/E).

16. The method of claim 15, wherein said therapeutically effective amount comprises an amount of between about 10⁻⁴ U/kg and about 35 U/kg.

17. The method of claim 15, wherein said therapeutically effective amount comprises an amount of between about 0.2 nanograms and about 2 nanograms.

18. The method of claim 15, wherein said method further comprises administration of a long-acting neurotoxin to the patient.

19. The method of claim 15, further comprising administering a botulinum toxin subtype A to the patient.

20. The method of claim 14, wherein said therapeutically effective amount comprises an amount that is below a dose that would cause muscle paralysis.
